(19) United States
(12) Patent Application Publication
(10) Pub. No.: US 2011/0172690 A1
(43) Pub. Date: Jul. 14, 2011

(54) Balloon Catheter for Use with a Surgical Coring System

(76) Inventor: William E. Cohn, Bellaire, TX (US)

(21) Appl. No.: 12/836,513

(22) Filed: Jul. 14, 2010

Related U.S. Application Data
(60) Provisional application No. 61/225,344, filed on Jul. 14, 2009.

(51) Int. Cl. A61B 17/32

(52) U.S. Cl. 606/170

(57) ABSTRACT

A balloon catheter for use with a surgical coring system for off pump surgery, includes various shaped balloon portions which are adapted to seal an apical hole in the left ventricle of a patient's heart.

### RELATED APPLICATION

This application claims the benefit, and priority benefit, of U.S. Patent Application Ser. No. 61/225,344, filed Jul. 14, 2009, entitled “Balloon Catheter For Use With a Surgical Coring System”.

### BACKGROUND

1. Field of the Disclosure

This disclosure relates generally to the field of surgery. More specifically, this disclosure relates to a balloon catheter for use with a surgical coring system. The balloon catheter and surgical coring system may be used for off-pump surgery.

2. Background of the Invention

In connection with the implantation of implantable left ventricular assist devices (“LVADs”), an apical hole is typically cored in the apex of the left ventricle of the patient's heart. Traditionally, the patient is connected to a heart-lung machine, usually referred to as cardiopulmonary bypass, during the LVAD implantation procedure. As discussed in U.S. Published Patent Application No. US 2008/0009891, now U.S. Pat. No. 7,744,527, entitled Surgical Coring Systems, doctors have begun exploring surgical techniques without the use of the heart-lung machine, or off-pump surgery.

### BRIEF SUMMARY

In accordance with an illustrative embodiment hereinafter described, the present balloon catheter for use with a surgical coring system may include two inflatable, expandable balloon portions for sealing the cored apical hole in the apex of the left ventricle and for releasably securing the balloon catheter in a sealed relationship with respect to the apical hole.

In accordance with another illustrative embodiment hereinafter described, the present balloon catheter may include a first inflatable, expandable balloon portion for sealing the cored apical hole in the apex of the left ventricle, and a second inflatable, expandable portion adapted to be disposed within the apical hole.

### BRIEF DESCRIPTION OF THE DRAWING

The present balloon catheter may be understood by reference to the following description taken in conjunction with the accompanying drawing, in which:

FIGS. 1-3 are partial cross-sectional views of a surgical coring tool and a balloon catheter illustrating the coring of an organ, such as a heart;

FIG. 4 is a partial cross-sectional view of a balloon catheter used in a surgical coring system;

FIG. 5 is a partial cross-sectional top view of an embodiment of the present balloon catheter;

FIG. 6 is a partial cross-sectional front view of the balloon catheter of FIG. 5;

FIG. 7 is a partial cross-sectional view of another embodiment of the present balloon catheter;

FIG. 8, is a partial cross-sectional view of another embodiment of the present balloon catheter; and

FIG. 9, is a partial cross-sectional view of another embodiment of the present balloon catheter.

While certain embodiments of the present balloon catheter will be described in connection with the preferred illustrative embodiments shown herein, it will be understood that it is not intended to limit the invention to those embodiments. On the contrary, it is intended to cover all alternatives, modifications, and equivalents, as may be included within the spirit and scope of the invention as defined by the appended claims. In the drawing figures, which are not to scale, the same reference numerals are used throughout the description and in the drawing figures for components and elements having the same structure, and primed reference numerals are used for components and elements having a similar function and construction to those components and elements having the same unprimed reference numerals.

### DETAILED DESCRIPTION OF THE SPECIFIC EMBODIMENTS

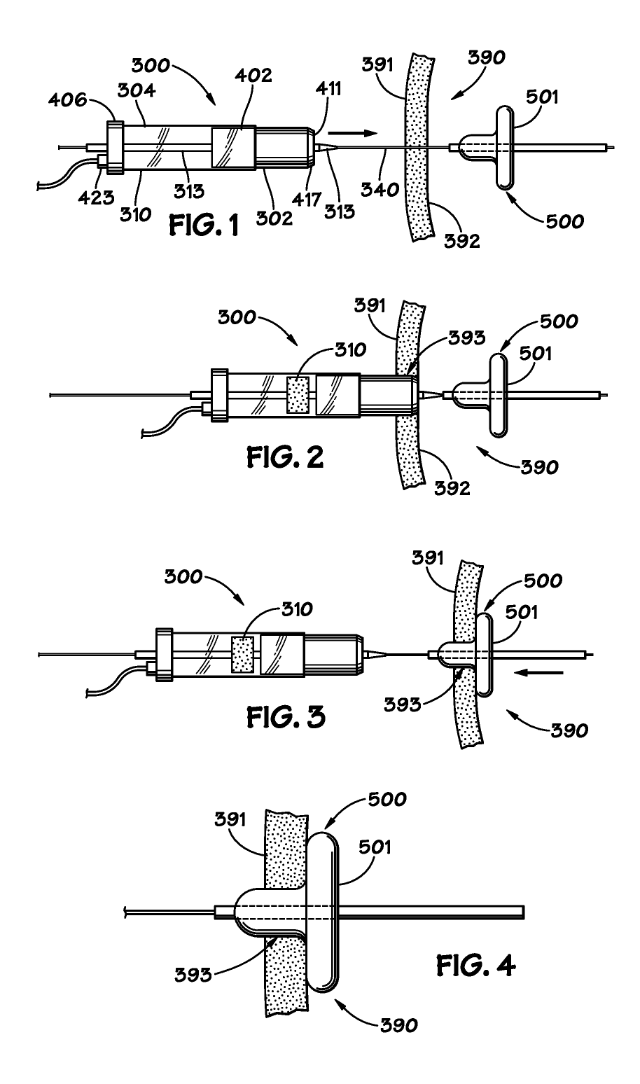

With reference to FIGS. 1-3, in connection with the implantation of an LVAD (not shown), and as discussed in U.S. Patent Application Publication No. US 2008/0009891, now U.S. Pat. No. 7,744,527, which is incorporated herein by reference, a coring tool 300 may be used to form a hole, or apical hole, 393 in the apex of the left ventricle of heart chamber 390, and a balloon catheter 500 may be utilized in combination with coring tool 300. As seen in FIG. 2, a section of heart tissue 310 has been removed from the wall 391, or apex of the left ventricle, of heart chamber 390, forming the hole, or apical hole, 393 in wall 391. With reference to FIGS. 2-4, as coring tool 300 is removed from the heart chamber 390, the inflated balloon 501 of balloon catheter 500 plugs the apical hole, or tissue cavity, 393 and a portion of the inflated balloon catheter 500 is assisted in moving into the apical hole 393 by the outward pressure created by the beating heart which includes heart chamber 390, as shown in FIGS. 3 and 4. Thus, balloon catheter 500 acts as a plug, or seal, to prevent the heart from spouting blood through the apical hole 393 left by excision, or coring, of the heart tissue 310. In addition, balloon catheter 500 is a further measure to prevent excised heart tissue 310 from falling back into the left ventricle of heart chamber 390.

An embodiment of a surgical coring system may include, coring tool 300 and a guide wire 340. Coring tool 300 may include: a hollow body 310; a vacuum chamber 304; an inner elongate member 313; and a coring portion 302 having a cutting edge 417. The coring portion 302 and vacuum chamber 304 may be formed integrally with each other and comprise a uniform hollow body 402 with an open distal end 411 and a closed proximal end 406. Closed proximal end 406 has a vacuum connection 423 to permit the application of suction, or a suction force, from the hollow body 402. Elongate member 313 is disposed coaxially within the hollow body 402 and extends past the open distal end 411, whereby the guide wire 340 may be received within the elongate member 313 and whereby the elongate member 313 is adapted to be inserted on to the guide wire 340.

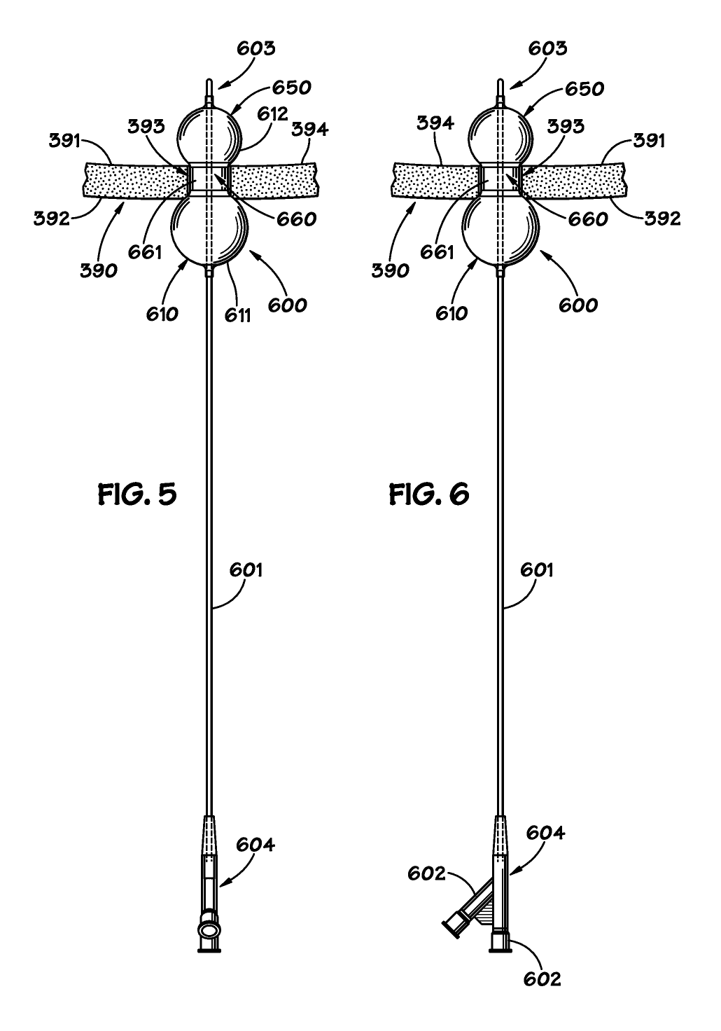

With reference to FIGS. 5 and 6, an embodiment of the present balloon catheter 600 is illustrated in its inflated, sealed configuration with respect to apical hole 393 formed in the wall 391, or apex of the left ventricle, of heart chamber 390. Balloon catheter 600 preferably has two inflatable, expandable balloon portions 610, 650 which as will hereinafter be described, are adapted for sealing the cored apical hole 393 in the wall 391, and for releasably securing the balloon catheter 600 in a sealed relationship with respect to the apical hole 393. Catheter 600 may include a conventional, flexible catheter body 601 adapted to be inserted through the femoral artery of the patient, whereby the uninflated (not shown) balloon portions 610, 650 may be initially disposed within heart chamber 390. Catheter 600 may also include conventional ports 602 for the passage of a conventional inflating fluid or gas for expanding balloon portion 610, 650, and for receipt of a conventional guide wire (not shown). The two expandable balloon portions 610, 650 are generally disposed at the distal end 603 of catheter body 601.

As seen in FIGS. 5 and 6, the first inflatable, expandable balloon portion 610 may have a generally spherical configuration when expanded and inflated as shown in FIGS. 5 and 6. The first inflatable, expandable balloon portion 610 may preferably be an inflatable, expandable balloon 611, which upon being inflated and expanded as shown in FIGS. 5 and 6, has a generally spherical configuration, and upon abutting the interior surface 392 of wall 391 of heart chamber 390 adjacent apical hole 393, seals the cored apical hole 393 to prevent the egress of blood (not shown) from within heart chamber 390 through apical hole 393.

Still with reference to FIGS. 5 and 6, the second inflatable, expandable balloon portion 650 of catheter 600 may also have a generally spherical configuration when expanded and inflated, as seen in FIGS. 5 and 6. Preferably the second inflatable, expandable balloon portion 650 is a second inflatable, expandable balloon 612, which upon inflation and expansion has the generally spherical configuration shown in FIGS. 5 and 6. Upon the inflation and expansion of the second balloon 612, at a location adjacent the outer wall surface 394 of wall 391 and adjacent apical hole 393, second balloon 612 serves to releasably secure balloon catheter 600 with respect to the apical hole 393 so that balloon catheter may not be pulled or moved back into heart chamber 390. Preferably balloon 612 serves to releasably secure balloon catheter 600 in a sealed relationship with respect to the apical hole 393. In this regard, after second balloon 612 has been inflated and expanded, as will be hereinafter described, as shown in FIGS. 5 and 6, the expanded balloon 612 will releasably secure the first balloon portion 610, or balloon 611, of catheter 600 in a sealed relationship with respect to the apical hole 393. Should a force be exerted upon catheter body 601 in the direction toward the proximal end 604 of balloon catheter 600, the inflated second balloon 612 would bear upon the outer wall surface 394 of wall 391 adjacent apical hole 393 to prevent the movement of the first balloon portion 610 away from its sealed relationship with respect to apical hole 393.

If desired, balloon catheter 600 may also include a third inflatable, expandable balloon portion 660 disposed intermediate the first and second balloon portions 610, 650. The third balloon portion 660 preferably has a generally cylindrical configuration and is sized to snugly fit within apical hole 393 upon inflation of the third expandable balloon portion 660. Preferably, the third balloon portion 660 is an inflatable, expandable balloon 661 having a generally cylindrical configuration as shown in FIGS. 5 and 6 and a generally circular cross-sectional configuration, when viewed along the catheter body 601, corresponding to the circular cross-sectional configuration of apical hole 393. If apical hole 393 has a non-circular cross-sectional configuration, such as square, hexagonal, oval, etc., the cross-sectional configuration of the third balloon portion 660 may be formed with a corresponding, mating cross-sectional configuration. Similarly, should apical hole 393 have a generally non-circular cross-sectional configuration, the first and second balloon portions 610, 650 could be provided with a similar, mating cross-sectional configuration upon inflation and expansion, or they could still be utilized with a spherical configuration.

If a third inflatable, expandable balloon portion 660 is utilized, it may be formed integral with the first balloon portion 610, and inflatable and expandable therewith, or alternatively, it may be provided with its own separate inflation and expansion capabilities, as by providing a suitable lumen, or passageway, (not shown) within catheter body 601 for the supply of an inflation liquid or gas, whereby the third balloon portion 660 may be independently expanded and inflated.

All of the expandable balloon portions 610, 650, 660 may be formed of any suitable material having the requisite ability to be inflated and expanded as shown in FIGS. 5 and 6, and the requisite characteristics to be utilized within the human body.

In operation, the first and second balloon portions 610, 650, and if utilized, third balloon portion 660 would be initially deployed in their unexpanded, or uninflated, configuration, to permit passage of catheter body 601 and balloon portions 610, 650, and 660 into the femoral artery of a patient and to a location within heart chamber 390 generally adjacent to the interior wall surface 392 of wall 391 of heart chamber 390, as is generally shown with respect to balloon catheter 500 in FIGS. 1 and 2. When balloon catheter 600 is disposed adjacent the interior wall surface 392 of heart chamber 390 prior to coring the apical hole 393, as shown in FIG. 2, or after the apical hole 393 is formed by coring tool 300, the first balloon portion 610 is expanded and inflated into the configuration illustrated in FIGS. 5 and 6. At this time the second balloon portion 650 remains in its deflated and unexpanded configuration. If the third balloon portion 660 is being utilized, it could also be expanded and inflated when the first balloon portion 610 is expanded and inflated.

Upon removal of the excised tissue 310, and the removal of coring tool 300 from apical hole 393, the expanded balloon portions 610 and if utilized balloon portion 660 would be passed into the apical hole 393 in the same manner as shown in FIGS. 3 and 4 with respect to catheter 500. After first balloon portion 610 is disposed in a sealing relationship with the apical hole 393 and bears against the inner wall surface 392 of heart chamber 390 adjacent apical hole 393, the second balloon portion 650 would be expanded and inflated as illustrated in FIGS. 5 and 6. If the third balloon portion 660 is utilized, the third balloon portion 650 could also be inflated and expanded after the third balloon portion 660 is received within apical hole 393. If as previously described, a force is exerted upon catheter body 601 to cause it to move in a direction toward the proximal end 604 of catheter 600, the second balloon portion 650 would bear against the outer wall surface 394 of wall 391 to secure, or restrain, the first balloon portion 610 from moving away from its sealed disposition with respect to apical hole 393. After a surgeon has associated an LVAD with respect to apical hole 393, the balloon portions 610, 650, and if utilized balloon portion 660, would be deflated so that balloon catheter 600 could be removed from the patient.

It should be noted that although the balloon catheter 600 has been described in connection with the use of a coring tool for coring an opening in the heart of a patient, it should be understood that the balloon catheter 600 could be utilized in connection with a coring tool operating upon other organs or blood vessels requiring resection of a defined portion of tissue, such as the bladder, stomach, liver, etc.

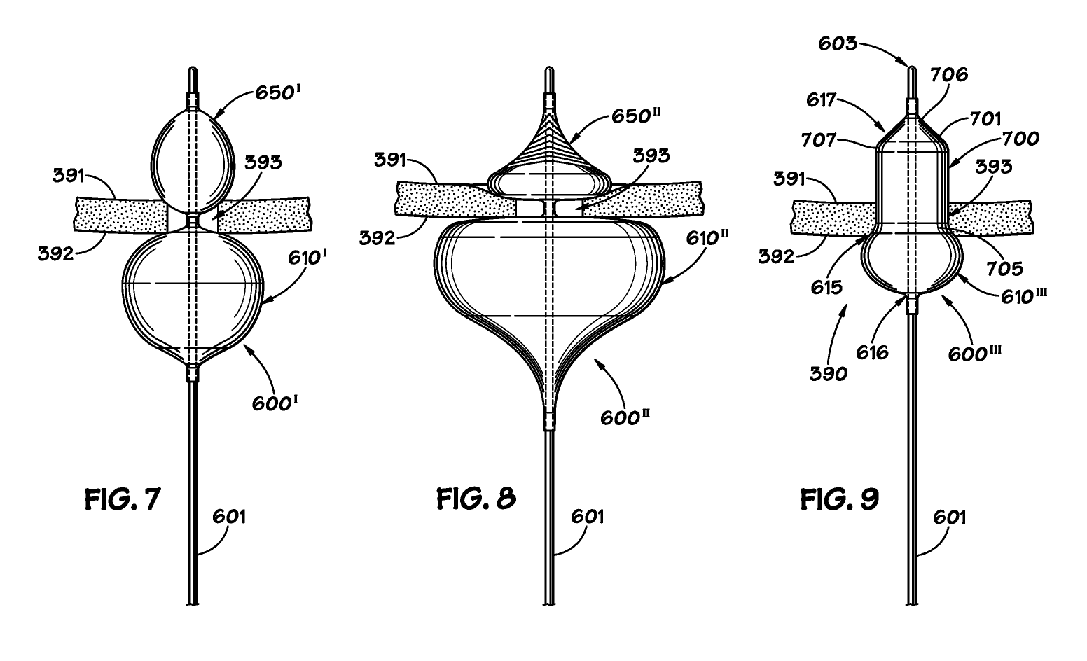

With respect to FIG. 7, another embodiment of a balloon catheter 600′ is illustrated. Catheter 600′ has first and second inflatable, expandable balloon portions 610′, 650′. Whereas the first and second balloon portions 610, 650 of catheter 600 have generally the same cross-sectional size and shape, the shape and diameter of the first and second balloon portions 610′, 650′ are different, and balloon catheter 600′ does not utilize the third balloon portion as found at 660 in catheter 600 of FIGS. 5 and 6. The balloon portions 610′, 650′ may have a generally spherical configuration, or an oval shaped configuration, or the shape of an ovoid, and the second balloon portion 650′ may be smaller in size than the first balloon portion 610′. The operation, construction, and utilization of balloon catheter 600′ is substantially the same as that previously described in connection with catheter 600 of FIGS. 5 and 6.

With respect to FIG. 8, another embodiment of a balloon catheter 600″ is illustrated, having first and second inflatable, expandable balloon portions 610″ and 650″. When the first and second balloon portions 610″ and 650″ are inflated and expanded as shown in FIG. 8, they may each have generally a frustoconical configuration as shown in FIG. 8, with a generally circular cross-sectional configuration when viewed along the catheter body, and the second balloon portion 650″ has a generally smaller diameter, or size, than the first balloon portion 610″. Again, the operation, construction, and utilization of catheter 600″ is substantially the same as that described in connection with the operation and utilization of catheter 600 of FIGS. 5 and 6.

With respect to FIG. 9, another embodiment of a balloon catheter 600′ is illustrated, having first and second inflatable, expandable balloon portions 610′″ and 700. When the first and second balloon portions 610′″ and 700 are inflated and expanded as shown in FIG. 9, the first balloon portion 610′″ has a generally oval or elliptical shaped configuration when viewed from the side of balloon portion 610′″ as shown in FIG. 9. The upper portion 615 of balloon portion 610′″ is removed, or truncated, so that the balloon portion 610′″ may also be considered as having a truncated, elliptical configuration as shown in FIG. 9. Upon the inflation and expansion of balloon portion 610′″, the upper portion 615 of balloon portion 610′″ abuts the interior surface 392 of wall 391 of heart chamber 390 or other organ (not shown), adjacent apical hole 393, and seals the cored apical hole 393 to prevent the egress of blood (not shown) from within the heart chamber 390, or other organ, through apical hole 393.

Still with respect to FIG. 9, the second inflatable, expandable balloon portion 700 preferably has a generally cylindrical configuration and is sized to snuggly fit within apical hole 393, upon inflation of the second expandable balloon portion 700. Second balloon portion 700 preferably has a generally circular cross-sectional configuration corresponding to the circular cross-sectional configuration of apical hole 393. If apical hole 393 has a non-circular cross-sectional configuration, such as square, hexagonal, oval, etc., the cross-sectional configuration of the second balloon portion 700 may be formed with a corresponding mating cross-sectional configuration. Preferably, the first and second expandable balloon portions 610′″ and 700 are formed integrally with each other and may form a single inflatable, expandable balloon 701, which may be easily formed, as by a conventional blow molding process. Balloon 701 has a generally cylindrical shaped balloon portion 700 disposed substantially adjacent to the distal end 603 of catheter body 601, and the first expandable balloon portion 610′″, having the generally elliptical cross-section configuration is disposed below the second balloon portion 700, toward the proximal end 616 of balloon 701.

Still with reference to FIG. 9, balloon 701 may include a curved, chamfered wall surface 705 disposed between the first and second balloon portions 610′″ and 700. The upper, or distal end 617 of balloon 701 preferably has a generally frustoconical wall surface 706, with a curved, chamfered wall surface 707 disposed between wall surface 706 and the generally cylindrical shaped balloon portion 700. The frustoconical wall surface 706 is believed to be advantageous when balloon catheter 600′″ is in its initially deflated configuration during delivery of balloon catheter 600′″ to the heart chamber 390. The generally elliptical shaped balloon portion 610′″ is also believed to be advantageous in making its delivery easier to the heart chamber 390. The height of the first balloon portion 610′″, as measured from the proximal end 616 of balloon 701 to the inner surface 392 of heart chamber 390 is preferably less than the heights of the other first balloon portions, 610′, 610″, which provide ease of delivery of balloon 701, as well as is easier to inflate into the desired configuration shown in FIG. 9. The spherical configuration of first balloon portion 610′″ also is believed to provide a sufficient seal for apical hole 393, including sufficient rigidity to maintain a sufficient sealing of apical hole 392 within heart chamber 390, as shown in FIG. 9. Preferably the length of the second balloon portion 700 is greater than the thickness of the body organ wall 391, whereby a balloon catheter 600′″ may be used in connection with body organ walls of different patients having different body organ wall thicknesses.

Specific embodiments of the present balloon catheter have been described and illustrated. It will be understood to those skilled in the art that changes and modifications may be made without departing from the spirit and scope of the inventions to be defined by the appended claims.

We claim:

1. A surgical coring system for coring an opening in a body organ comprising:
a guide wire;
a coring tool comprising
a hollow body having an open distal end and a closed proximal end, the open distal end having a cutting edge, wherein the hollow body comprises a vacuum connection to apply suction from the hollow body; and
an elongate member disposed coaxially within the hollow body and extending past the open distal end, wherein the guidewire may be received within the elongate member; and
a balloon catheter adapted to be coaxially mounted on to the guidewire, the balloon catheter including a catheter body having a distal end and at least a first and a second inflatable, expandable balloon portion associated with the catheter body adjacent the distal end of the catheter body, the at least first and second inflatable, expandable balloon portions being spaced apart from each other along the catheter body, and upon inflation of the at least first and second inflatable, expandable balloon portions, each of the at least first and second balloon portions have a diameter greater than the diameter of the coring tool, whereby the at least first balloon portion may seal the hole in the body organ, and the at least second balloon portion may releasably secure the balloon catheter with respect to the opening in the body organ.

2. The surgical coring system of claim 1, wherein the at least first inflatable, expandable balloon portion has a generally spherical configuration

3. The surgical coring system of claim 2, wherein the at least second inflatable, expandable balloon portion has a generally spherical configuration.

4. The surgical coring system of claim 2, wherein the at least second inflatable, expandable balloon portion has a generally ovoid shape.

5. The surgical coring system of claim 1, wherein the at least first inflatable, expandable balloon portion has a generally frustoconical configuration.

6. The surgical coring system of claim 5, wherein the at least second inflatable, expandable balloon portion has a generally frustoconical configuration.

7. The surgical coring system of claim 1, including a third inflatable, expandable balloon portion disposed intermediate the at least first and second inflatable, expandable balloon portions.

8. The surgical coring system of claim 7, wherein the third inflatable, expandable balloon portion has a generally cylindrical configuration.

9. The surgical coring system of claim 8, wherein third inflatable, expandable balloon portion is adapted to be disposed within the opening in the body organ.

10. A balloon catheter for use with a surgical coring system for coring an opening in a body organ comprising:
an elongate catheter body having a distal end and a first and a second inflatable, expandable balloon portion disposed adjacent the distal end of the catheter body;
the first balloon portion having a generally elliptical configuration and a substantially circular cross-sectional configuration; and
the second balloon portion has a generally cylindrical configuration.

11. The balloon catheter of claim 10, wherein the first and second balloon portions are formed integral with each other.

12. The balloon catheter of claim 10, wherein upon inflation and expansion of the first balloon portion, the first balloon portion has a diameter which is greater than the opening in the body organ.

13. The balloon catheter of claim 10, wherein upon inflation and expansion of the second balloon portion, the second balloon portion has a diameter substantially equal to the diameter of the opening in the body organ.

14. The balloon catheter of claim 10, wherein a curved, chamfered wall surface is disposed between the first and second balloon portions.

15. The balloon catheter of claim 10, wherein a curved, chamfered wall surface is disposed adjacent a distal end of the second balloon portion.

16. The balloon catheter of claim 15, wherein the second balloon portion has a distal end having a generally frustoconical configuration, and the curved chamfered wall surface is disposed between the distal end of the second balloon portion and a portion of the second balloon portion having the cylindrical configuration.

17. The balloon catheter of claim 10, wherein the body organ has a thickness adjacent the opening, and the second balloon portion has a length which is greater than the thickness of the body organ.

18. A surgical coring system for coring an opening in a body organ comprising:
a guide wire;
a coring tool comprising a hollow body having an open distal end and a closed proximal end, the open distal end having a cutting edge, wherein the hollow body comprises a vacuum connection to apply suction from the hollow body; and
a balloon catheter adapted to be coaxially mounted on to the guidewire, the balloon catheter having
an elongate catheter body having a distal end and a first and a second inflatable, expandable balloon portion disposed adjacent the distal end of the catheter body; the first balloon portion having a generally elliptical configuration and a substantially circular cross-sectional configuration and the second balloon portion has a generally cylindrical configuration.

19. The balloon catheter of claim 18, wherein the first and second balloon portions are formed integral with each other.

20. The balloon catheter of claim 18, wherein upon inflation and expansion of the first balloon portion, the first balloon portion has a diameter which is greater than the opening in the body organ.

21. The balloon catheter of claim 18, wherein upon inflation and expansion of the second balloon portion, the second balloon portion has a diameter substantially equal to the diameter of the opening in the body organ.

22. The balloon catheter of claim 18, wherein a curved, chamfered wall surface is disposed between the first and second balloon portions.

23. The balloon catheter of claim 18, wherein a curved, chamfered wall surface is disposed adjacent a distal end of the second balloon portion.

24. The balloon catheter of claim 23, wherein the second balloon portion has a distal end having a generally frustoconical configuration, and the curved chamfered wall surface is disposed between the distal end of the second balloon portion and a portion of the second balloon portion having the cylindrical configuration.

25. The balloon catheter of claim 18, wherein the body organ has a thickness adjacent the opening, and the second balloon portion has a length which is greater than the thickness of the body organ.
